(19) United States
(12) Patent Application Publication
(10) Pub. No.: US 2024/0327934 A1
(43) Pub. Date: Oct. 3, 2024

(54) ANALYSIS DEVICE AND ANALYSIS METHOD

(71) Applicant: CANON MEDICAL SYSTEMS CORPORATION, Tochigi (JP)

(72) Inventors: Xiaotian WU, Beijing (CN); Hao HUANG, Beijing (CN); Qiqi XU, Beijing (CN)

(73) Assignee: CANON MEDICAL SYSTEMS CORPORATION, Tochigi (JP)

(21) Appl. No.: 18/621,634

(22) Filed: Mar. 29, 2024

(30) Foreign Application Priority Data
Mar. 31, 2023 (CN) 202310339586.3
Mar. 15, 2024 (JP) 2024-040673

(51) Int. Cl. C12Q 1/70; B01L 3/00; C12Q 1/6844

(52) U.S. Cl. CPC C12Q 1/701; B01L 3/5023; C12Q 1/6844; B01L 2300/069; B01L 2300/0825; B01L 2300/161

(57) ABSTRACT

The analysis device according to an embodiment includes a partition region including a water-soluble partition material capable of individually partitioning between reaction regions different from each other in the device.

### REFERENCE TO A SEQUENCE LISTING

In accordance with 37 CFR § 1.833-1835 and 37 CFR § 1.77 (b) (5), the specification makes reference to a Sequence Listing submitted electronically as a .xml file named “552774US_ST26.xml”. The .xml file was generated on Mar. 29, 2024 and is 9,371 bytes in size. The entire contents of the Sequence Listing are hereby incorporated by reference.

### CROSS-REFERENCE TO RELATED APPLICATIONS

This application is based upon and claims the benefit of priority from Chinese Patent Application No. 202310339586.3, filed on Mar. 31, 2023; and Japanese Patent Application No. 2024-040673, filed on Mar. 15, 2024, the entire contents of all of which are incorporated herein by reference.

### FIELD

Embodiments described herein relate generally to an analysis device and an analysis method.

### BACKGROUND

Development of CRISPR technology provides a possibility of quick, accurate, highly sensitive, and convenient detection of viral nucleic acids, and becomes popular technology in a point-of-care testing (POCT) diagnosis field. Herein, Cas12 and Cas13 proteins have a large application potential in detection of an in vitro nucleic acid molecule. Cas12 targets double-stranded DNA (dsDNA), and Cas13 targets single-stranded RNA (SSRNA). These two types of Cas proteins (also called Cas enzymes) both have collateral cleavage activity in an in vitro environment, and the activity thereof belongs to cleavage activity of nonspecific nuclease. When a target nucleic acid molecule is targeted, the Cas protein is activated to acquire activity of nonspecifically cleaving such an optional nucleic acid molecule. By using such a point, a Specific High Sensitivity Enzymatic Reporter UnLOCKing (SHERLOCK) nucleic acid detection system based on Cas13a and a DNA Endonuclease Targeted CRISPR Trans Reporter (DETECTR) nucleic acid detection system based on Cas12a have been respectively designed.

A method for detecting nucleic acids using the SHERLOCK nucleic acid detection system and the DETECTR nucleic acid detection system mainly includes three steps. First, by using an isothermal nucleic acid amplification technique (for example, Recombinase Polymerase Amplification (RPA), loop-mediated isothermal amplification (LAMP), and the like), or performing isothermal nucleic acid amplification after reverse transcription (for example, at the time of detecting an RNA virus), content of nucleic acids in a sample is stably amplified; next, the amplified sample is directly reacted with Cas12a (DETECTR) or reacted with Cas13a (SHERLOCK) after transcription, and in a case in which a target nucleic acid molecule is present in the sample, the Cas protein is bound with the target nucleic acid by guide crRNA guidance, cleavage activity of nonspecific nuclease thereof is activated, and a reporter nucleic acid molecule is further cleaved; and finally, the sample after the reaction is completed is dropped onto a dedicated test strip, and presence/absence of the cleaved reporter nucleic acid molecule in a reaction product is detected to determine whether the target nucleic acid molecule is present in the sample.

For example, WO2021/149829 discloses a method for refining a sample, and detecting a specific DNA in the sample using a side-flow detection test strip after performing LAMP amplification.

However, such a method for detecting nucleic acids requires a plurality of steps such as nucleic acid extraction, amplification, a Cas enzyme reaction, and test strip detection, operation is complicated, there is a risk of contamination due to repetition of lid-opening operation during these steps, dedicated devices and operators are required, and the operation is hardly performed by an individual, so that the method needs to be improved in view of integration and automation.

Paper-based lateral flow test (LFT) is a diagnosis scheme that is quick and easily used, but can typically complete only a single reaction with one piece of paper. To implement integration and automatic detection, that is, implement a reaction by connecting a plurality of reactions in series on one piece of paper, a flow of a reaction liquid is typically delayed by using a special substance. Although sequential reactions can be automatically performed with these methods, there are problems such that accurate control is yet hardly performed, design is complicated, cost is high, versatility is low, and the like.

For example, Barry Lutz, et al., “Dissolvable fluidic time delays for programming multi-step assays in instrument-free paper diagnostics”, Lab Chip, Vol. 13 (14), 2013, pages 2840-2847 discloses a method for detecting a methylated DNA using LFT technology in which a fluid time is delayed from several minutes to one hour by adding sucrose solutions having different concentrations (10 to 70% of saturation degree) to a paper strip to be dried, speeds of different reaction solutions to reach the center are adjusted, the different reaction solutions are sequentially mixed, and sequential reactions are achieved. However, the device is required to constantly adjust sucrose concentrations in different reaction regions to be matched with an appropriate reaction time. A high sucrose concentration may influence activity of an enzyme and a reaction speed, so that a flowing time can be delayed by only one hour at the maximum at the same time.

Additionally, Peter Q. Nguyen, et al., “Wearable materials with embedded synthetic biology sensors for biomolecule detection”, Nature Biotechnology, Vol. 39, 2021, pages 1366-1374 discloses a wearable material that implements a sequential flow of liquid using a folding system, controls delay of time by adjusting concentration of polyvinyl alcohol (PVA), and can noninvasively detect SARS-CoV-2, for example. However, in this method, manufacture needs to be performed by a special printing device in addition to preparing a test paper, and the PVA concentration and a use amount needs to be constantly adjusted for different reaction demands, so that it is difficult to accurately control the time.

A problem to be solved by the present invention is to provide an analysis device and an analysis method that can easily perform an examination including a plurality of enzyme reactions.

The analysis device according to an embodiment includes a partition region including a water-soluble partition material capable of individually partitioning between reaction regions different from each other in the device.

### BRIEF DESCRIPTION OF THE DRAWINGS

FIG. 1 is a flowchart at the time of manufacturing and using an analysis device according to an embodiment;

FIG. 2 is a schematic diagram of a state of dissolution of a partition region (exemplifying PVA) at the time of using the analysis device manufactured in the present embodiment;

FIG. 3A is a schematic diagram of a layer configuration of the analysis device;

FIG. 3B is a schematic diagram (plan view) of an external appearance of the analysis device;

FIG. 4 is a schematic diagram illustrating a state of a solution flow at the time of using the analysis device according to an embodiment;

FIG. 5A is a layer configuration diagram of the analysis device;

FIG. 5B is a side view (upper row) and a plan view (lower row) of the external appearance of the analysis device;

FIG. 5C is a side view (upper row) and a plan view (lower row) of an inner part of the analysis device;

FIG. 6 is a schematic diagram illustrating a state of a solution flow at the time of using the analysis device according to another embodiment;

FIG. 7 represents photographs at the time when 2 minutes and 3 minutes have elapsed after sufficiently dropping ink on the analysis device of an example 1 and adding water on PVA;

FIG. 8 represents photographs immediately after immersing one end of the analysis device in the example 1 in the ink, and 3 hours later;

FIG. 9 represents a photograph at the time of dissolving a first PVA region in which LAMP-Cas12a enzyme fragmentation detection is performed on a target nucleic acid (SARS-CoV2) using an analysis device of an example 2; and

FIG. 10 represents photographs at the time of completing LAMP-Cas12a enzyme fragmentation detection performed on the target nucleic acid (SARS-CoV2) using the analysis device of the example 2.

### DETAILED DESCRIPTION

Next, the following describes a specific embodiment of the present embodiment in detail with reference to the drawings. The following description about the embodiment is made for describing a concept of the present embodiment, and does not intend to limit the present embodiment.

The present embodiment aims at providing an analysis device that solves a problem of highly accurate control of a plurality of steps of enzyme reaction in an In Vitro Diagnosis (IVD) detection process.

As a result of vigorous investigation on problems in a conventional technique, the inventors of the present application have found that the problem about highly accurate control of the device can be solved by changing an operation of a partition region in the conventional technique from a mode of “delay” to a mode of “completely ON/completely OFF”.

The present embodiment can be applied to various kinds of IVD detection requiring an enzyme reaction step, used together with a loading device, add each reagent quantitatively at a regular time due to automatic loading by the loading device, and further achieve accurate control of automation of the detection method according to the present embodiment accordingly.

According to the present embodiment, it is possible to provide an analysis device that can be accurately controlled and is used for a plurality of sequential enzyme reactions.

The present embodiment exemplifies the analysis device that performs a reaction (enzyme reaction) in a solution infiltrated into a paper base (test paper), but the embodiment is not limited thereto. For example, a base material made of an optional material such as glass fiber can be applied instead of paper so long as the solution can be infiltrated therein by capillary action and a reaction can be performed.

### Analysis Device

The present embodiment relates to the analysis device including a partition region including a water-soluble partition material capable of individually partitioning between reaction regions different from each other in the device. Preferably, the partition region composed of the water-soluble partition material.

The water-soluble partition material may be any water-soluble material that can form a partition region completely partitioned on a base material, and is not particularly limited. In view of cost and simplicity of operation, it is preferable to use polyvinyl alcohol (PVA) or sucrose in the present embodiment.

The PVA is a water-soluble polymeric reagent, which can be dissolved in the base material under a liquid condition and can form a dense solid material (PVA region) after being dried. Due to this, an upstream liquid hardly directly passes through a cross section thereof, and after an aqueous solution is brought into contact therewith at a large area and dissolves a dense structure thereof, the solution can smoothly pass therethrough to a reaction region on a downstream side.

Sucrose has high solubility in aqueous solutions, can form a dense solid material (sucrose region) after being dried similarly to the PVA, and exhibits an effect of completely block a liquid flow.

The analysis device according to the present embodiment is preferably a side-flow detection test strip, but is not limited thereto. The analysis device according to the present embodiment may be another device used for in-situ chromogenic analysis, for example, detection of fluorescence excitation, color development due to an enzyme reaction (for example, Horseradish peroxidase (HRP)), and the like.

The base material used for the analysis device according to the present embodiment is not particularly limited. A typical material for manufacturing a test strip in a conventional technique can be used, for example, one type selected from a glass cellulose film, a cellulose nitrate film, and polyester cellulose film may be used. As the base material according to the present embodiment, a commercial product may be directly used, for example, a glass cellulose film Ahlstrom 8964 produced or represented by Shanghai Jieyi Biotechnology Co., Ltd., a polyester cellulose film MA 0280, a product of a water absorbing pad (Fusion) series, and the like.

A reaction device in the present embodiment can be used for various IVD detection methods requiring a plurality of enzyme reaction steps, and is not particularly limited. For example, the reaction device can be used for nucleic acid detection (for example, SHERLOCK nucleic acid detection or DETECTR nucleic acid detection) in which an isothermal amplification reaction and a reaction based on a Cas enzyme are sequentially performed in different reaction regions, immunity detection requiring sequential operation such as ELISA, and the like. In the present embodiment, the reaction device is preferably used for nucleic acid detection in which an isothermal amplification reaction and a reaction based on a Cas enzyme are sequentially performed in different reaction regions.

In the present embodiment, a separation region in a mode of “completely ON/completely OFF” can be manufactured by dissolving a water-soluble material such as PVA and sucrose in the base material, and the base material can be partitioned into different reaction regions due to the separation region.

The different reaction regions in the reaction device according to the present embodiment may be connected in series, or may be connected by superimposition. That is, the reaction regions are not necessarily separated by the partition region of PVA or sucrose on a common test strip, but the reaction regions may be each manufactured on a single base material and overlapped with each other by a PVA or sucrose region. In other words, the reaction regions different from each other may be formed by a common base material. Alternatively, the reaction regions different from each other may be individually formed by base materials different from each other, and at least part of the base materials may be superimposed on each other.

A shape of the analysis device according to the present embodiment is not particularly limited, and may be a quadrangular shape, a cube shape, a circular shape, or a cylindrical shape, for example.

### Method for Manufacturing Analysis Device

The following describes a method for manufacturing the analysis device according to the present embodiment.

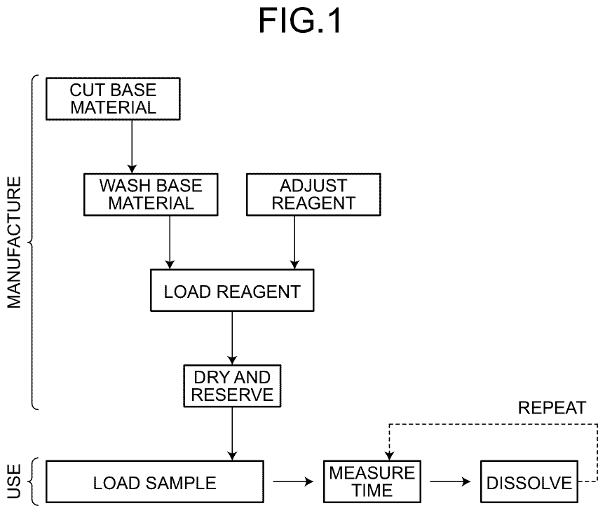

FIG. 1 is a flowchart at the time of manufacturing and using the analysis device according to the present embodiment.

As illustrated in FIG. 1, the method for manufacturing the analysis device according to the present embodiment can include steps of cutting a paper base, washing the paper base, preparing a reagent, loading the reagent, drying and reserving, and the like. Specifically, the base material (paper base) is cut to have appropriate shape and size, impurities on a paper rod is removed by washing processing, a water-soluble material having appropriate concentration, for example, a PVA or sucrose solution, is prepared, the solution is applied to an appropriate position of the base material, and the base material is partitioned and thereafter dried to be reserved for later use.

At the time of preparing the reagent, the concentration of the water-soluble material may be, for example, 5 to 60%, preferably 10 to 30%, and more preferably 15 to 25% by weight percentage.

At the time of loading the reagent, in view of forming the partition region in the mode of “completely ON/completely OFF” according to the present embodiment, a loading amount of the water-soluble material on the base material is preferably 5 to 40 μL/0.1 cm², 10 to 30 μL/0.1 cm², and more preferably 15 to 25 μL/0.1 cm² for the base material per unit area.

In view of forming the partition region in the mode of “completely ON/completely OFF” according to the present embodiment, an aspect ratio of a region of the dense solid material of the water-soluble material formed on the base material (that is, a ratio between a length in a longitudinal direction and a width in a cross-sectional direction of the base material in the partition region) may be equal to or larger than 1:1, preferably 1.5:1 to 5:1, and more preferably 2:1 to 4:1.

At the time of drying, a method for drying is not particularly limited. For example, the base material may be caused to stand still to be naturally dried, or may be put in an oven, a drying box, or the like to be dried.

Additionally, the analysis device according to the present embodiment can further bind a corresponding reaction reagent to a paper material in a different reaction region in advance.

In a case of binding the reaction reagent in advance, a scheme of binding the reaction reagent is not particularly limited, and a general scheme in this field can be employed. For example, stable reagents at the ordinary temperature can be directly isolated in a liquid form in independent chambers respectively, the reagents that have been subjected to freeze-drying, for example, bound to the base material in a form of freeze-dried microspheres or freeze-dried powder. In other words, the reaction reagents used in the respective reaction regions are loaded in advance on the base material in the respective reaction regions by freeze-drying.

At the time of detection, to prevent contamination of environment and evaporation of the reaction reagent, the method for manufacturing the analysis device according to the present embodiment may further include a sealing step, that is, a step of sealing the analysis device to be dried and reserved as described above with a sealing material. A specific scheme of sealing is not particularly limited, and a general sealing method for a test strip in this field can be employed. For example, the analysis device may be sealed in a plastic case, or in a plastic film.

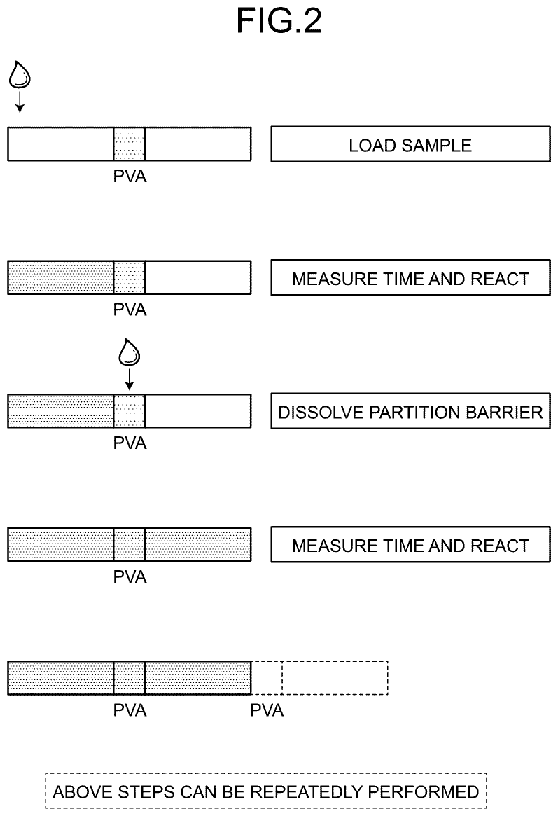

FIG. 2 is a schematic diagram of a state of dissolution of the partition region (exemplifying PVA) at the time of using the analysis device manufactured in the present embodiment.

As illustrated in FIG. 2, after a sample to be measured and a reaction reagent for a first region (as needed) are added to the first region of the reaction device in the present embodiment, an enzyme reaction proceeds in the first region, and at this point, the reaction solution is limited to be within the first region due to barrier working of the PVA partition region. At this point, although the solution in the sample is brought into contact with the PVA partition region, a contact surface between the solution and the PVA partition region is small, that is, only a cross-section portion of the PVA partition region, so that the entire PVA partition region is not dissolved in the solution in the sample. After the time is measured, the enzyme reaction in the first region is performed, and the reaction is ended, water or an aqueous solution and a reaction agent for a second region (as needed) are added to an upper surface of the PVA partition region. At this point, the entire PVA partition region (partition barrier) is dissolved in the solution, the solution after the reaction is completed in the first region passes through the PVA partition region and enters the second region accordingly, and an enzyme reaction in the second region proceeds. Due to this, it is possible to implement sequential enzyme reactions with the test paper according to the present embodiment.

In other words, even if the sample is dropped on one reaction region (first reaction region) of the reaction regions different from each other (the first reaction region and a second reaction region), the partition region can prevent the solution from being infiltrated into the other one of the different reaction regions. The partition region is dissolved when water or an aqueous solution is added to the upper surface of the partition region. As a result, the sample is infiltrated into the second reaction region from the first reaction region, so that the enzyme reaction in the second reaction region can be performed.

The number of the reaction regions disposed in the reaction device according to the present embodiment may be multiple, for example, two, three, or more in accordance with an actual demand for detection, and the different reaction regions are partitioned by the partition region according to the present embodiment. Herein, water-soluble materials used for the partition regions may be the same, or may be different from each other. That is, steps of “dissolving barrier—measuring time and reacting” in FIG. 2 may be set as a plurality of cycles as needed. In other words, the analysis device is used for sequential reactions.

### Analysis Device Using Isothermal Amplification Reaction and Cas Enzyme Reaction

As described above, the paper-based reactor according to the present embodiment can be used for various IVD detection methods requiring a plurality of enzyme reaction steps. The following describes, as an example, an analysis device used for nucleic acid detection in which an isothermal amplification reaction and a reaction based on a Cas enzyme are sequentially performed.

The analysis device includes, for example, a sample loading region, an isothermal amplification reaction region, a Cas enzyme reaction region, and a result reading region in order, and the isothermal amplification reaction region and the Cas enzyme reaction region, and the Cas enzyme reaction region and the result reading region are partitioned by partition regions according to the present embodiment, respectively In other words, the analysis device performs the isothermal amplification reaction and the reaction based on the Cas enzyme in order in reaction regions different from each other.

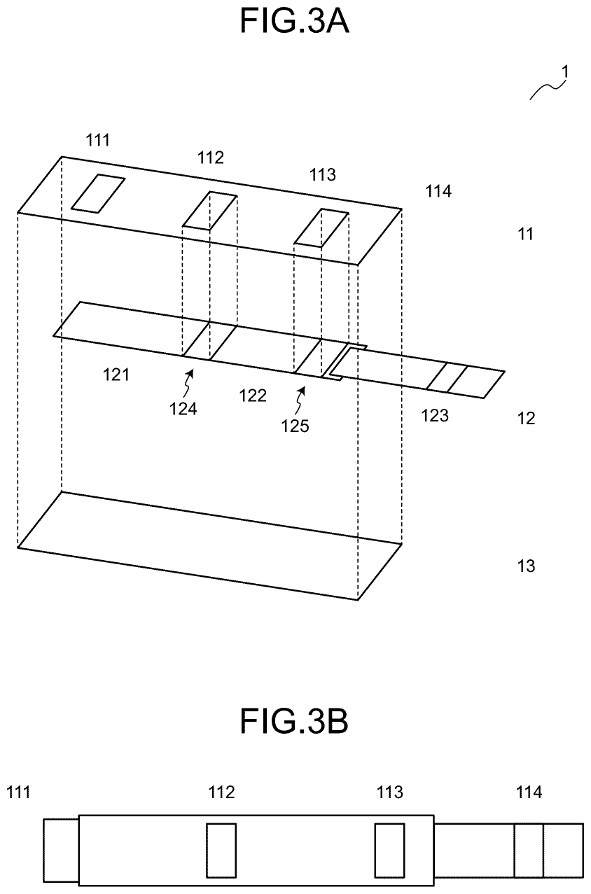

FIG. 3 is a schematic diagram of a configuration of the analysis device according to the embodiment. FIG. 3A is a schematic diagram of a layer configuration of the analysis device, and FIG. 3B is a schematic diagram (plan view) of an external appearance of the analysis device.

As illustrated in FIG. 3A, an analysis device 1 according to the embodiment includes three layers, that is, an upper part sealing layer 11, an analysis layer 12, and a lower part sealing layer 13 in order from top to bottom. The upper part sealing layer 11 includes a sample loading port 111, an opening 112, an opening 113, and a result reading window 114 in order. The analysis layer 12 includes an isothermal amplification reaction region 121, a Cas enzyme reaction region 122, and a result reading region 123 in order. Herein, the isothermal amplification reaction region 121 and the Cas enzyme reaction region 122, and the Cas enzyme reaction region 122 and the result reading region 123 are partitioned by partition regions 124 and 125 according to the present embodiment, respectively. In other words, the analysis device includes openings (the opening 112 and the opening 113) on an upper side of the partition regions.

In the isothermal amplification reaction region 121, an isothermal amplification reaction reagent may be bound to the base material in advance.

The isothermal amplification reaction may be RPA or LAMP, for example, and is not particularly limited. These isothermal amplification techniques have high sensitivity, high specificity, and rapid reactions, and do not require a temperature cycle device, so that these techniques are often applied to biological detection in POCT.

The RPA is a technique for implementing nucleic acid amplification under an isothermal condition in which a plurality of types of enzymes and proteins are involved. The RPA technique mainly depends on three types of enzymes, that is, a recombinant enzyme that can be bound with a single-stranded nucleic acid (oligonucleotide primer), a single-stranded DNA binding protein (SSB), and a strand-displacement DNA polymerase. A mixture of the three types of enzymes has activity even at the ordinary temperature, and amplification is completed under an isothermal condition (about 37 to 40° C.) within 20 to 40 minutes.

An RPA reaction reagent may include, for example, a recombinant enzyme, a single-stranded DNA binding protein (SSB) and a strand-displacement DNA polymerase, RPA Primer Mix, a template DNA or RNA, a reaction buffer solution, and the like. As the RPA reaction reagent, a commercial product, for example, TwistAmp® series RPA kit may be used as it is, and may be used for manufacturing the paper-based reactor according to the present embodiment after the component described above is appropriately prepared.

The LAMP is a novel isothermal nucleic acid amplification method. This technique uses four or six primers that can identify six specific regions on a target gene, depends on strong strand displacement activity of a strand-displacement DNA polymerase, and completes amplification under an isothermal condition (about 60 to 65° C.) within 30 to 60 minutes.

A LAMP reaction reagent may include, for example, a strand-displacement DNA polymerase, dNTP, LAMP Primer Mix, a template DNA or RNA, a reaction buffer solution, and the like. As the LAMP reaction reagent, commercial products, for example, a LAMP kit manufactured by Shanghai LMAI Bio, and WarmStart® fluorescent LAMP/RT-LAMP kit manufactured by New England Biolabs may be used as they are, and may be used for manufacturing the paper-based reactor according to the present embodiment after the components described above are appropriately prepared.

In the Cas enzyme reaction region, a Cas enzyme, a guide nucleic acid, a reporter nucleic acid, a reaction buffer solution, and the like may be bound to the base material.

The Cas enzyme includes at least one type of Cas enzyme selected from Cas12 (VA type), Cas13 (VI type), and Cas14 (VF type). The Cas enzyme may have cleavage activity for a target nucleic acid, and is at least one type selected from Cas12a, Cas13a, Cas13b, Cas14a, Cas14b, and Cas14c, for example. The Cas enzyme does not necessarily have cleavage activity for a target nucleic acid, and is at least one type selected from dCas12a, dCas13a, dCas13b, dCas14a, dCas14b and dCas14c, for example.

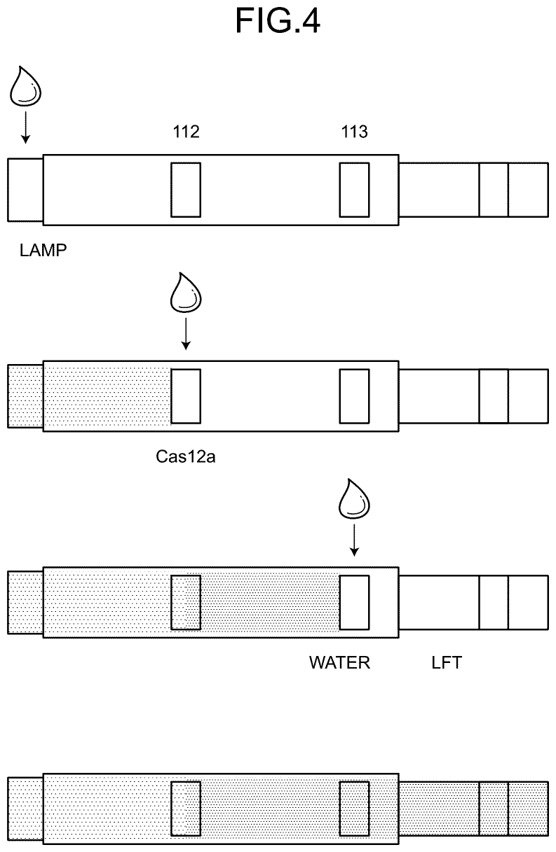

FIG. 4 is a schematic diagram illustrating a state of a solution flow at the time of using the analysis device according to the embodiment.

As illustrated in FIG. 4, in a case of use, the sample to be measured is added through the sample loading port 111, water or an aqueous solution is added through the opening 112 after the isothermal amplification reaction, for example, the LAMP reaction is completed, the water-soluble material, for example, PVA in the partition region 124 is dissolved, the solution after the LAMP reaction is completed enters the Cas enzyme reaction region 122, water or an aqueous solution is further added through the opening 113 after the Cas enzyme reaction is completed, the partition region 125 is dissolved, the solution after the Cas enzyme reaction is completed enters the result reading region 123, and a result is expressed.

In the analysis device according to the present embodiment, the isothermal amplification reaction reagent and/or the Cas enzyme reaction reagent does not necessarily bound in advance. For example, the isothermal amplification reaction reagent may be added to a sample solution to be measured and added through the sample loading port 111 together with the sample to be measured. The Cas enzyme reaction reagent is prepared in an aqueous solution to be added through the opening 112, dissolves PVA in the partition region 124, and enters the Cas enzyme reaction region 122 to react.

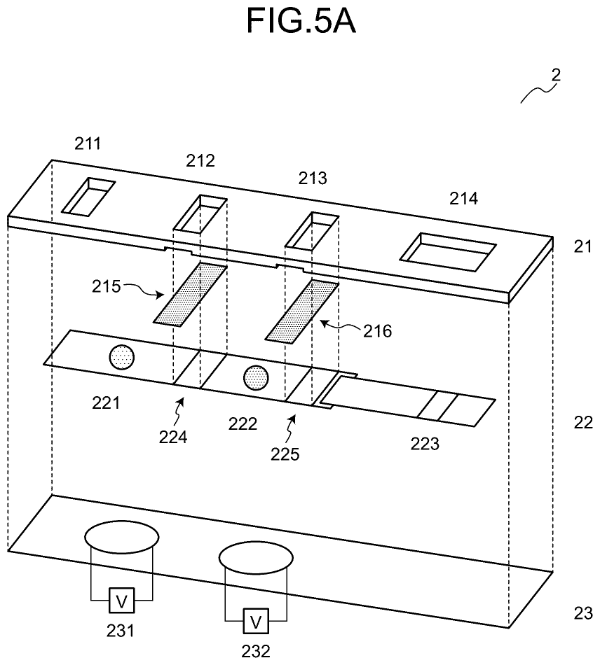

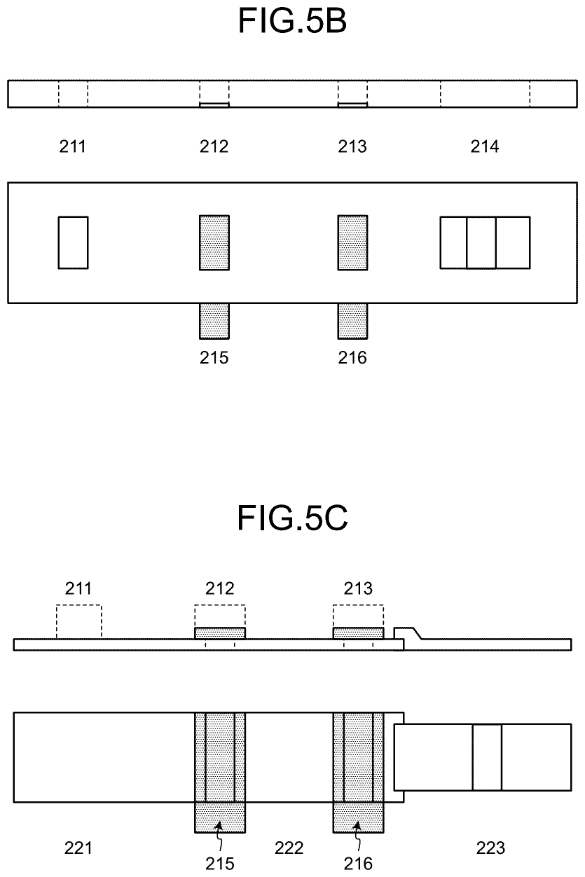

FIG. 5 is a schematic diagram of the configuration of the analysis device according to another embodiment. FIG. 5A is a diagram of a layer configuration of the analysis device, FIG. 5B is a side view (upper row) and a plan view (lower row) of the external appearance of the analysis device, and FIG. 5C is a side view (upper row) and a plan view (lower row) of the inner part of the analysis device.

As illustrated in FIG. 5, an analysis device 2 according to the other embodiment includes three layers, that is, an upper part sealing case layer 21, an analysis layer 22, and a lower part sealing case layer 23 in order from top to bottom. The upper part sealing case layer 11 includes a sample loading port 211, a chamber 212, a chamber 213, and a result reading window 214 in order. The analysis layer 22 includes an isothermal amplification reaction region 221, a Cas enzyme reaction region 222, and a result reading region 223 in order. Herein, the isothermal amplification reaction region 221 and the Cas enzyme reaction region 222, and the Cas enzyme reaction region 222 and the result reading region 223 are partitioned by partition regions 224 and 225 according to the present embodiment, respectively.

The chamber 212 and the chamber 213 each have a well that can house liquid, and bottom parts of the chamber 212 and the chamber 213 are sealed by hydrophobic film 215 and 216 (hydrophobic strips), respectively. Each of the chamber 212 and the chamber 213 can house water or an aqueous solution for dissolving the partition region. In a case of dissolving the partition region, the hydrophobic film 215 or 216 is pulled out to enable the solution in the chamber 212 or the chamber 213 to enter a corresponding partition region of the analysis layer on a lower side, and the partition region can be dissolved accordingly. In FIG. 5A, dots in the isothermal amplification reaction region 221 and the Cas enzyme reaction region 222 in the analysis layer 22 indicate the reaction reagents that have been bound to the respective reaction regions in advance. However, in a case in which the corresponding reaction reagents are not bound to the respective reaction regions in the analysis layer 22 in advance as described above, the sample to be measured and the solution in the chamber 212 may include the corresponding reaction reagent. In other words, the reaction reagents used in the respective reaction regions are loaded in a liquid state into the chambers disposed on an upper side of the respective reaction regions.

In other words, the analysis device includes a housing region (chamber) that houses water or an aqueous solution on the upper side of the partition region, and a hydrophobic support member (hydrophobic film) that supports water or an aqueous solution so that water or an aqueous solution does not flow out into the partition region, the hydrophobic support member being able to be pulled out. Due to this, a user can add water or an aqueous solution to the upper surface of the partition region by pulling out the hydrophobic film.

In the lower part sealing case layer 23, heaters 231 and 232 are disposed at positions on the lower side corresponding to the isothermal amplification reaction region 221 and the Cas enzyme reaction region 222, and can supplementarily heat the isothermal amplification reaction region 221 and the Cas enzyme reaction region 222 as needed.

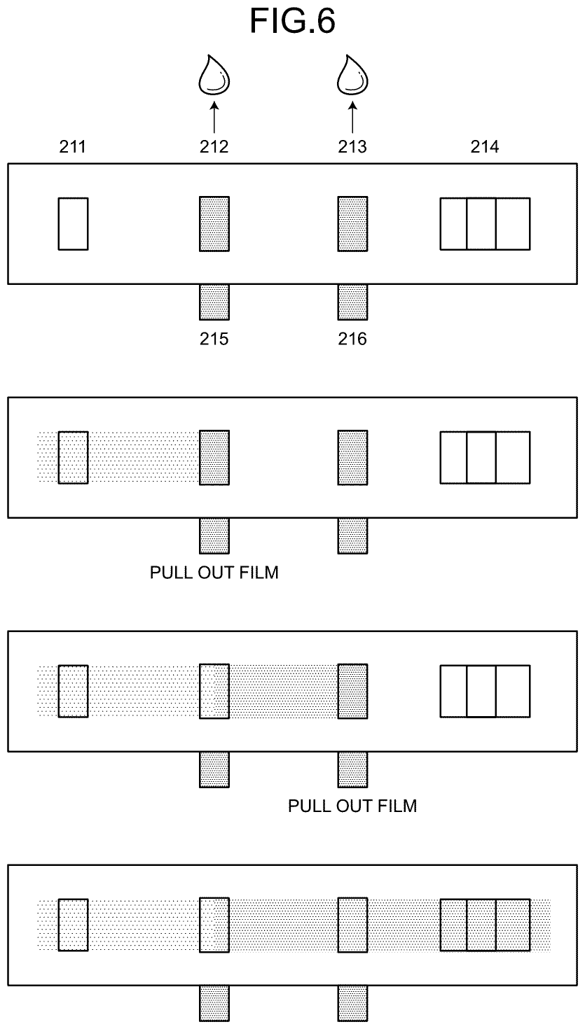

FIG. 6 is a schematic diagram illustrating a state of a solution flow at the time of using the analysis device according to another embodiment.

As illustrated in FIG. 6, after the isothermal amplification reaction is completed, the hydrophobic film 215 is pulled out to cause the solution in the chamber 212 to flow to the partition region 224 of the analysis layer on the lower side and dissolve the partition region 224, the solution after the isothermal amplification reaction is completed enters the Cas enzyme reaction region 222, the hydrophobic film 216 is pulled out to cause the solution in the chamber 213 to flow to the partition region 225 of the analysis layer on the lower side and dissolve the partition region 225 after the Cas enzyme reaction is completed, and the solution after the Cas enzyme reaction is completed enters the result reading region 223, and a result is expressed accordingly.

The analysis device according to the present embodiment can be used for detecting a target molecule in a sample to be detected. The target molecule may be a nucleic acid, protein, and the like, and the nucleic acid is not limited to a nucleic acid of a virus, but may be various nucleic acids to be detected. The sample to be detected is also not particularly limited, but may be snivel, saliva, urine, blood, blood serum, cerebrospinal fluid, or the like, for example.

The analysis device and the manufacturing method therefor according to the present embodiment, applications thereof, and the like have been described above, but the present embodiment is not limited thereto. The present embodiment encompasses a form obtained by applying various modifications conceivable by those skilled in the art to the embodiment, and another form constructed by combining some of the constituent elements in the embodiment without departing from the gist of the present embodiment.

The present embodiment may also be provided as an analysis method using the analysis device described above. That is, in the analysis method, in the analysis device including the partition region made of the water-soluble partition material capable of partitioning between the different reaction regions in the device, water or an aqueous solution is added to the upper surface of the partition region to dissolve the partition region. Due to this, the analysis method enables examinations including a plurality of enzyme reactions to be easily performed.

### EXAMPLES

The following specifically describes paper-based analysis according to the present embodiment using examples, but the present embodiment is not limited to the examples.

### Example 1

In this example, the analysis device according to the present embodiment (hereinafter, also referred to as a “test strip”) is manufactured and an ink test is performed, and steps thereof are described below.

### 1. Cutting Paper Base

A Fusion3 water absorbing pad (SHANGHAI JIYI) is cut in an appropriate length (4.5 cm×0.2 cm).

### 2. Washing Paper Base

1) A cut Fusion3 paper strip is put in a centrifuge tube of 50 mL, and washed three times with Nuclease-Free Water (Thermo Fisher/Invitrogen™ (Ambion™), AM9930, the same applies to the following), each time for 10 minutes.
2) Next, after 50 mL of 5% (w/v) bovine serum albumin (BSA) is added and the Fusion3 paper strip is shaken and incubated for two hours, it is washed three times with Nuclease-Free Water, each time for 10 minutes.
3) Nuclease-Free Water containing 4% (v/v) RNAsecure is added, and the Fusion3 paper strip is incubated for 20 minutes at 60° C.
4) The Fusion3 paper strip is washed three times with Nuclease-Free Water thereafter, each time for 10 minutes.
5) Finally, the Fusion3 paper strip is put in a centrifuge tube of 1.5 mL, and dried for 20 minutes at 80° C. by employing a vacuum high-temperature centrifugation method.

### 3. Preparing Reagent

PVA powder (Mowiol@8-88) is added to Nuclease-Free Water to be prepared to a 20% solution, and dissolution is accelerated by using an ultrasonic water bath.

### 4. Reagent Loading and Drying

20 μL of the dissolved PVA solution is dropped onto the middle (a region from 2 cm to 2.5 cm) of the cut test strip. After the test strip sufficiently absorbs the PVA solution, the test strip is put in a blast drying box at 50° C. to be dried, manufactured to be the analysis device of the example 1, and placed at a dry cool place to be reserved until being used. A test strip on which the PVA solution is not dropped is assumed to be a negative control.

### 5. Time Measurement Dissolution

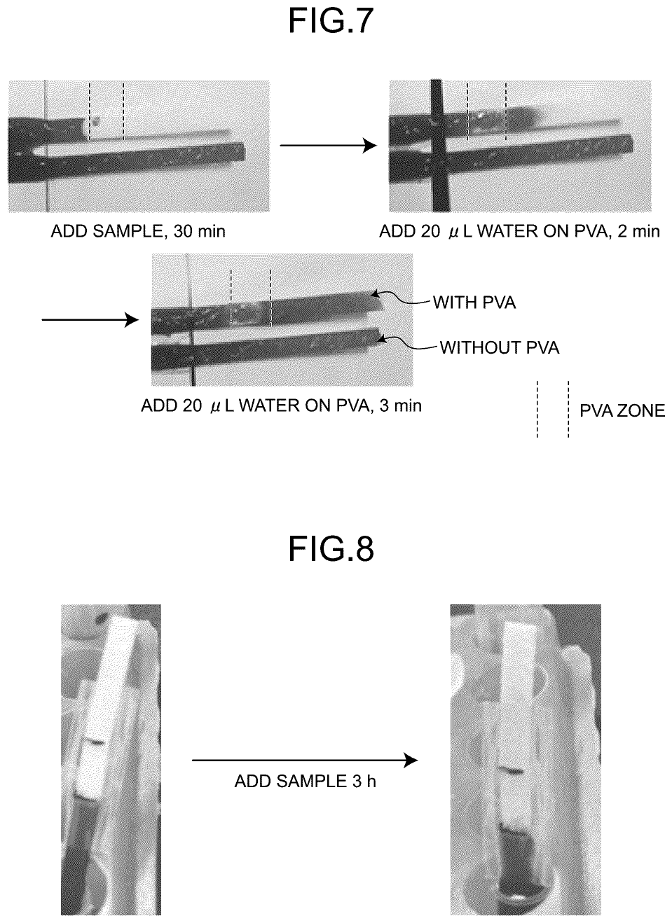

FIG. 7 represents photographs at the time when 2 minutes and 3 minutes have elapsed after sufficiently dropping ink on the analysis device of the example 1 and adding water on the PVA.

As illustrated in FIG. 7, a sufficient amount of ink was added to the first region to sufficiently infiltrate for 30 minutes. At this point, it was observed that the ink was limited to the first region and did not pass through the PVA region. Next, 20 μL of water was dropped on the PVA region, and it was observed that the ink flowed from the first region to the second region to infiltrate into the second region when about two minutes had elapsed, and the ink completely infiltrated into the second region when three minutes had elapsed. On the other hand, regarding the negative control (the same test strip not including the PVA region), after the ink was dropped on one end of the test strip, the ink directly infiltrated into the entire test strip.

FIG. 8 represents photographs immediately after immersing one end of the analysis device of the example 1 in the ink, and 3 hours later.

As illustrated in FIG. 8, in the analysis device according to the present embodiment, the PVA region and the second region are hardly changed after the one end is immersed in the ink for 3 hours, and the ink is limited to the first region.

As is clear from the example 1, according to the present embodiment, the PVA region disposed on the test strip can completely separate the test strip into two regions, that is, front and rear regions. A liquid in the front region hardly influences a form of the PVA region, but after a liquid is added from the upper surface of the PVA region to dissolve the PVA region, the solution in the front region enters and infiltrates into the second region in a short time.

### Example 2

In this example, the analysis device (hereinafter, also referred to as a “test strip”) based on the LAMP isothermal amplification reaction and the Cas enzyme reaction is manufactured, and a gene of SARS-CoV2 is detected by the device. Specific manufacturing and detection steps are as follows.

### 1. Cutting Paper Base

A Fusion3 water absorbing pad (SHANGHAI JIYI) is cut in an appropriate length (5.5 cm×0.2 cm).

### 2. Washing Paper Base

1) The cut Fusion3 paper strip is put in the centrifuge tube of 50 mL, and washed three times with Nuclease-Free Water, each time for 10 minutes.
2) Next, after 50 mL of 5% (w/v) bovine serum albumin (BSA) is added and the Fusion3 paper strip is shaken and incubated for two hours, it is washed three times with Nuclease-Free Water, each time for 10 minutes.
3) Nuclease-Free Water containing 4% (v/v) RNAsecure is added, and the Fusion3 paper strip is incubated for 20 minutes at 60° C.
4) The Fusion3 paper strip is washed three times with Nuclease-Free Water thereafter, each time for 10 minutes
5) Finally, the Fusion3 paper strip is put in a centrifuge tube of 1.5 mL, and dried for 20 minutes at 80° C. by employing a vacuum high-temperature centrifugation method.

### 3. Preparing Reagent

PVA powder (Mowiol@8-88) is added to Nuclease-Free Water to be prepared to a 20% solution, and dissolution is accelerated by using an ultrasonic water bath.

### 4. Reagent Loading and Drying

20 μL of the dissolved PVA solution is dropped onto the middle (a section from 2 cm to 2.5 cm and 4.5 to 5 cm) of the cut test strip. After the test strip sufficiently absorbs the PVA solution, the test strip is put in a blast drying box at 50° C. to be dried, and placed at a dry cool place to be reserved until being used. Next, the first reaction region is wrapped with tin foil paper and sealed with PCR tape to be used for an isothermal LAMP reaction.

### 5. Detection Use

A solution containing the LAMP reaction reagent (a positive control contains a target nucleic acid (SARS-CoV2), but the negative control does not contain it) is dropped on a front reaction section. After only the section portion is placed on an iron block in a metal bath at 65° C. to react for 45 minutes, a solution containing the Cas12a reaction reagent is dropped on the PVA region to react for 45 minutes at the ordinary temperature or 37° C., water is dropped on a second PVA region, and an end thereof is connected to an LFT detection test strip in series.

Herein, the solution containing the LAMP reaction reagent is prepared to 50 μL of reaction solution using WarmStart® fluorescent LAMP/RT-LAMP kit (containing UDG) (New England Biolabs (NEB), #E 1708S, 100rxns) in accordance with an operation protocol of the kit, which mainly includes WarmStart LAMP 2× Master Mix, Primer Mix, a template (that is, the target nucleic acid (SARS-CoV2)), and the like.

The solution containing the Cas12a reaction reagent is prepared to 50 μL of reaction solution using EnGen® Lba Cas12a (Cpf1) nuclease kit (New England Biolabs (NEB), #M 0653T, 2,000 pmoles) in accordance with an operation protocol of the kit, which mainly includes LbCas12a, NEB buffer 2.1, Cas12a-crRNA, and a probe (FITC/FAM-T 12-Biotin).

Respective nucleic acid sequences of the target nucleic acid SARS-CoV2 (N gene) and the Cas12a-crRNA are indicated by the following Table 1, and primers to be used are indicated by the following Table 2.

TABLE 1

| Se- | | |
|---|---|---|
| quence | Gene | |
| number | name | Sequence |
| 1 | SARS- | ATGTCTGATAATGGACCCCAAAATCAGCGAAAT |
| | CoV2 | GCACCCCGCATTACGTTTGGTGGACCCTCAGATT |
| | | CAACTGGCAGTAACCAGAATGGAGAACGCAGTG |
| | | GGGCGCGATCAAAACAACGTCGGCCCCAAGGTT |
| | | TACCCAATAATACTGCGTCTTGGTTCACCGCTCT |
| | | CACTCAACATGGCAAGGAAGACCTTAAATTCCCT |
| | | CGAGGACAAGGCGTTCCAATTAACACCAATAGC |
| | | AGTCCAGATGACCAAATTGGCTACTACCGAAGA |
| | | GCTACCAGACGAATTCGTGGTGGTGACGGTAAA |
| | | ATGAAAGATCTCAGTCCAAGATGGTATTTCTACT |
| | | ACCTAGGAACTGGGCCAGAAGCTGGACTTCCCT |
| | | ATGGTGCTAACAAAGACGGCATCATATGGGTTG |
| | | CAACTGAGGGAGCCTTGAATACACCAAAAGATC |
| | | ACATTGGCACCCGCAATCCTGCTAACAATGCTGC |
| | | AATCGTGCTACAACTTCCTCAAGGAACAACATTG |
| | | CCAAAAGGCTTCTACGCAGAAGGGAGCAGAGGC |
| | | GGCAGTCAAGCCTCTTCTCGTTCCTCATCACGTA |
| | | GTCGCAACAGTTCAAGAAATTCAACTCCAGGCA |
| | | GCAGTAGGGGAACTTCTCCTGCTAGAATGGCTGG |
| | | CAATGGCGGTGATGCTGCTCTTGCTTTGCTGCTG |
| | | CTTGACAGATTGAACCAGCTTGAGAGCAAAATG |
| | | TCTGGTAAAGGCCAACAACAACAAGGCCAAACT |
| | | GTCACTAAGAAATCTGCTGCTGAGGCTTCTAAGA |
| | | AGCCTCGGCAAAAACGTACTGCCACTAAAGCAT |
| | | ACAATGTAACACAAGCTTTCGGCAGACGTGGTCC |
| | | AGAACAAACCCAAGGAAATTTTGGGGACCAGGA |
| | | ACTAATCAGACAAGGAACTGATTACAAACATTG |
| | | GCCGCAAATTGCACAATTTGCCCCCAGCGCTTC |
| | | AGCGTTCTTCGGAATGTCGCGCATTGGCATGGA |
| | | AGTCACACCTTCGGGAACGTGGTTGACCTACACA |
| | | GGTGCCATCAAATTGGATGACAAAGATCCAAAT |
| | | TTCAAAGATCAAGTCATTTTGCTGAATAAGCATA |
| | | TTGACGCATACAAAACATTCCCACCAACAGAGC |
| | | CTAAAAAGGACAAAAAGAAGAAGGCTGATGAA |
| | | ACTCAAGCCTTACCGCAGAGACAGAAGAAACAG |
| | | CAAACTGTGACTCTTCTTCCTGCTGCAGATTTGG |
| | | ATGATTTCTCCAAACAATTGCAACAATCCATGAG |
| | | CAGTGCTGACTCAACTCAGGCCTAA |
| 2 | Cas12a- | UAAUUUCUACUAAGUGUAGAUUUGAACUGUUG |
| | crRNA | CGACUACGU |

TABLE 2

| Sequence | | |
|---|---|---|
| number | Primer | Sequence |
| 3 | F3 | GCTGCAATCGTGCTACAACT |
| 4 | B3 | TCTGTCAAGCAGCAGCAAAG |
| 5 | FIP-2 | TGCGACTACGTGATGAGGAACGTTGCCAAAAG |
| | | GCTTCTACGC |
| 6 | BIP-2 | TTCAACTCCAGGCAGCAGTAGGCAAGAGCAGC |
| | | ATCACCGC |
| 7 | LF-2 | TTGACTGCCGCCTCTGC |
| 8 | LB-2 | GGAACTTCTCCTGCTAGAATGGC |

A method for preparing a primer mixture (primer mix) is as follows.

Custom primer dry powder was dissolved in Nuclease-Free Water, and prepared to a 100 μM Stock solution. Next, 100 μL of LAMP primer mix solution was prepared with volumes indicated by the following Table 3.

TABLE 3

| Primer | Volume (μL) | |
|---|---|---|
| FIP-2 | 16 | μL |
| BIP-2 | 16 | μL |
| F3 | 2 | μL |
| B3 | 2 | μL |
| LF-2 | 4 | μL |
| LB-2 | 4 | μL |
| Water | 56 | μL |
| Total | 100 | μL |

### 6. Output of Results

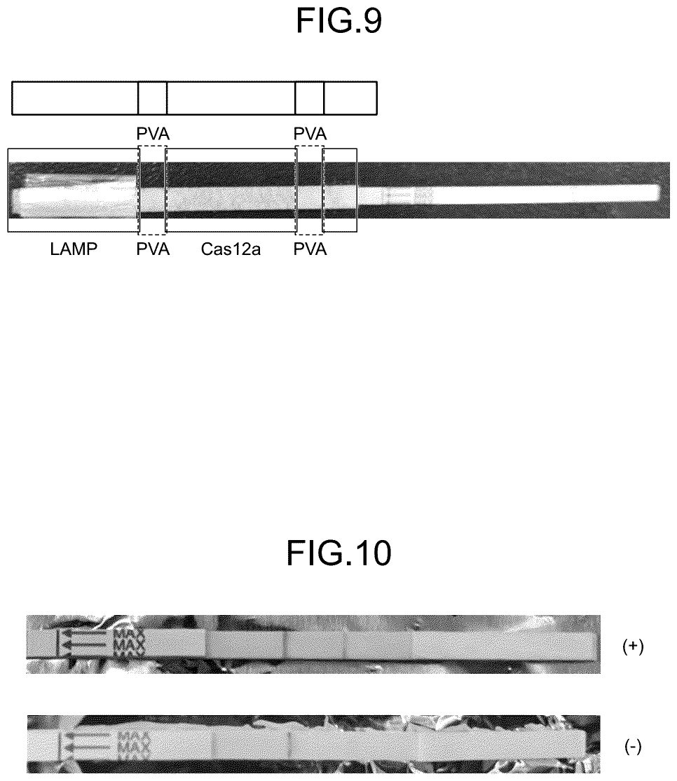

FIG. 9 represents a photograph at the time of dissolving a first PVA region in which LAMP-Cas12a enzyme fragmentation detection is performed on the target nucleic acid (SARS-CoV2) using the analysis device of the example 2.

FIG. 10 represents photographs at the time of completing LAMP-Cas12a enzyme fragmentation detection performed on the target nucleic acid (SARS-CoV2) using the analysis device of the example 2.

As illustrated in FIGS. 9 and 10, the liquid in the LAMP reaction region hardly influences a form of the PVA region. After the liquid was added to dissolve the PVA region, the liquid in the front region entered in a short time and infiltrated into the second reaction region, and two strips were represented in the positive control (+).

As described above, according to the present embodiment, it is possible to provide the analysis device that can be accurately controlled and is used for a plurality of sequential enzyme reactions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An analysis device comprising:
a partition region including a water-soluble partition material capable of individually partitioning between reaction regions different from each other in a device.

2. The analysis device according to claim 1, wherein the water-soluble partition material is polyvinyl alcohol (PVA) or sucrose.

3. The analysis device according to claim 1, wherein a loading amount of the water-soluble partition material on a base material is 10 to 30 μL/0.1 cm² for the base material per unit area.

4. The analysis device according to claim 1, wherein an aspect ratio of the partition region is equal to or larger than 1:1.

5. The analysis device according to claim 1, wherein the partition region is dissolved when water or an aqueous solution is added to an upper surface of the partition region.

6. The analysis device according to claim 1, wherein an isothermal amplification reaction and a reaction based on a Cas enzyme are performed in order in the reaction regions different from each other.

7. The analysis device according to claim 1, wherein reaction reagents used in respective reaction regions are loaded in advance on a base material in the respective reaction regions by freeze-drying.

8. The analysis device according to claim 1, wherein reaction reagents used in respective reaction regions are loaded in a liquid state into chambers disposed on an upper side of the respective reaction regions.

9. The analysis device according to claim 1, wherein a base material is any one of a glass cellulose film, a cellulose nitrate film, and a polyester cellulose film.

10. The analysis device according to claim 1, wherein the device has a quadrangular shape, a cube shape, a circular shape, or a cylindrical shape.

11. The analysis device according to claim 1, wherein the device is sealed in a plastic case or a plastic film.

12. The analysis device according to claim 1, wherein the reaction regions different from each other are formed by a common base material.

13. The analysis device according to claim 1, wherein the reaction regions different from each other are individually formed by base materials different from each other, and at least part of the base materials may be superimposed on each other.

14. The analysis device according to claim 1, further comprising an opening on an upper side of the partition region.

15. The analysis device according to claim 1, further comprising:
a housing region configured to house water or an aqueous solution on an upper side of the partition region; and
a hydrophobic support member configured to support the water or the aqueous solution so that the water or the aqueous solution does not flow out into the partition region, the hydrophobic support member being able to be pulled out.

16. The analysis device according to claim 1, wherein, even in a case where a sample is dropped on one reaction region of the reaction regions different from each other, the partition region is able to prevent a solution from being infiltrated into the other one of the different reaction regions.

17. The analysis device according to claim 1, used for sequential reactions.

18. An analysis method performed by an analysis device including a partition region made of a water-soluble partition material capable of partitioning between different reaction regions in a device, the analysis method comprising dissolving the partition region by adding water or an aqueous solution to an upper surface of the partition region.


SEQUENCE LISTING (8 sequences)

SEQ ID NO: 1 - DNA, 1260 bp, SARS-CoV-2
  source 1..1260: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 2 - RNA, 41 nt, synthetic construct
  source 1..41: mol_type = other RNA
taatttctactaagtgtagatttgaactgttgcgactacgt

SEQ ID NO: 3 - DNA, 20 bp, synthetic construct
  source 1..20: mol_type = other DNA
gctgcaatcgtgctacaact

SEQ ID NO: 4 - DNA, 20 bp, synthetic construct
  source 1..20: mol_type = other DNA
tctgtcaagcagcagcaaag

SEQ ID NO: 5 - DNA, 42 bp, synthetic construct
  source 1..42: mol_type = other DNA
tgcgactacgtgatgaggaacgttgccaaaaggcttctacgc

SEQ ID NO: 6 - DNA, 40 bp, synthetic construct
  source 1..40: mol_type = other DNA
ttcaactccaggcagcagtaggcaagagcagcatcaccgc

SEQ ID NO: 7 - DNA, 17 bp, synthetic construct
  source 1..17: mol_type = other DNA
ttgactgccgcctctgc

SEQ ID NO: 8 - DNA, 23 bp, synthetic construct
  source 1..23: mol_type = other DNA
ggaacttctcctgctagaatggc